(19) United States
(12) Patent Application Publication
(10) Pub. No.: US 2002/0022052 A1
(43) Pub. Date: Feb. 21, 2002

(54) Transdermal delivery system

(76) Inventor: Charles William Dransfield, Lake Cathie (AU)

Correspondence Address: Paul F. McQuade, GREENBERG TRAURIG, 12th FLOOR, 1750 TYSONS BLVD., MCLEAN, VA, 22102, US

(21) Appl. No.: 09/863,764

(22) Filed: May 24, 2001

(30) Foreign Application Priority Data
Jul. 21, 2000 (AU) PQ8885
Apr. 6, 2000 (AU) PQ6691

(51) Int. Cl. A61K 9/70

(52) U.S. Cl. 424/449

(57) ABSTRACT

A transdermal or transepithelial composition and a method for making a transdermal or transepithelial composition substantially free of water comprising a biologically active agent in the form of microfined particles, sized less than 2 microns down to less than 0.1 microns, which by massage pressure are mechanically entrained within the interstices of the stratum corneum. Particles less than 0.5 microns do not require a carrier for entrainment. Delivery into mucosal epithelia is obtained by particles less than one micron with delivery increasing with decreasing particle size.

### REFERENCE TO RELATED APPLICATIONS

The present application claims priority, under the Paris Convention for the Protection of Industrial Property, stemming from Provisional Australian Patent Application Serial Number PQ8885, filed Jul. 21, 2000 with the Australian Patent Office. The teachings of Provisional Australian Patent Application Serial Number PQ6691, filed Apr. 6, 2000, and Provisional Australian Patent Application Serial Number PQ8885, filed Jul. 21, 2000 are incorporated herein by reference in their entirety.

### FIELD OF THE INVENTION

The present invention relates generally to transdermal drug delivery, and more particularly relates to methods of delivery and the composition of biologically active agents which allow or enhance transdermal or transepithelial delivery of drugs into the body.

### BACKGROUND OF THE INVENTION

Increasing the efficiency and effectiveness of the administration of pharmaceutical drugs and biologically active agents into the human body is of vast importance to patients, doctors, and pharmaceutical companies. The present invention as disclosed provides an improved transport method of delivery and composition for the administration of biologically active agents through transdermal delivery. The present invention's effectiveness has been derived and premised upon an under appreciated epithelial structure, as will be discussed in more detail below.

The ability to deliver biologically active agents through the skin and mucous epithelia has many advantages over the normal gastro-intestinal route. Where skin, joint, muscle and mucous linings of the body cavities are the targets the biologically active agents can be introduced directly into the problem area entailing a lower overall dose, the sparing of side effects which accompany oral administration, assured absorption, and maintenance of the structure of actives unaffected by gastrointestinal digestion and absorption limitations. It has recently been found that the structure of keratinised epithelial tissue that constitutes the skin is unique.

While the present invention covers epithelial tissue in general, differences in technique and usage may be required when considering either keratinised or non-keratinised epithelium. To keep the boundaries clear, delivery involving the skin is nominated as transdermal delivery and delivery in the epithelia of the gastro intestinal tract, mouth, respiratory system, nasal cavities, lungs, vagina and rectum are designated epithelial delivery. The mode of transdermal absorption using water containing transdermal products and skin patch technology highlights both the carrying mechanism and the limitations inherent in these forms of delivery.

Prior tests using ascorbic acid as the active agent (in a water based admixture) have shown that the mechanics of absorption and the limited delivery rate of water based transdermal products have provided a linear absorption rate. The tests were conducted over a period of 72 hours and showed a maximum percentage of ascorbic acid absorbed in the 72 hour time period of 12.4% (mean value) of the applied dose of ascorbic acid. A hypothesis given for this delivery as stated in the Journal of Cutaneous Aging and Cosmetic Dermatology, Volume 1, Nov. 2, 1988/89 in an article by Kaplan et al states: “The effect of ionization and of different temperatures on tendencies of polar and non polar compounds has resulted in the hypothesis of a separate continuous polar shunt in the stratum corneum. The exact character of the polar pathway remains to be elucidated but the apparent existence of this route of penetration explains the ability of ascorbic acid to permeate through the skin.”

A number of other patch technologies have shown similar time rate delivery curves, such as U.S. Pat. No. 4,983,382 by Willmott et al. The Willmott et al patent further confirms the limiting figures of delivery as the studies quoted produce almost identical delivery results as Kaplan et al. vis 12.8% (mean value) absorption of actives over 72 hours.

Studies have also shown and documented that there is a fluid flow circuit incorporated in the skin structure which carries 7-dehydrocholesterol through the stratum corneum to the surface for conversion by UV radiation into cholecalciferol (a vitamin D precursor) and then re-absorbs the converted fatty acid. Further studies have shown a second circuit in the skin structure which reticulates oils and fats to be modified into hormone precursors which are then reabsorbed. These circuits herein are referred to as the ‘reticulatory circuits’. While the mechanism postulated for the polar shunt is still not confirmed, the presence of these other reticulatory/absorption circuits systems is well documented.

While the effectiveness of the present invention has been demonstrated, its exact mechanism is not certain, but is premised on the theory that the reticulatory circuits are the primary mechanism utilized in all transdermal delivery systems. The generally linear nature of transdermal absorption (confirmed in studies presented herewithin) and the consistent rate and mass limitation of both patch and liquid based transdermal absorption appears to confirm this as the mechanism.

Patch technology relies on the “reticulatory circuits” for its ability to get the material through the keratinised cells of the stratum corneum. Patches are structured to hold a selected active agent against the skin long enough for some of it to be dissolved in, and picked up, along with the reticulating substances and taken into the system. This liquid delivery system is restricted as to both quantity and rate by the essentially small volume of materials reticulated through the highly keratinised outer cells and the slow operating rate of the system and by the restrictive surface area of the material exposed to this transport mechanism.

The use of plastic wrap (occlusion) over topical creams duplicates patch technology. Transdermal delivery systems that do not use patches but rely on water, water/oil and oil/water based carriers also depend on the “reticulatory circuits” for delivery. In the transdermal delivery systems relying on water based carriers the amount of material absorbed and the rate of delivery are further limited by the homeostatic border sealing of the stratum corneum being triggered by the water content of the carrier. It is also obvious that oil based mixtures which are left adhering to the skin will be delivered over time by the reticulatory circuits.

Keratinised stratum corneum cells are dead cells that have no biochemical activity. These cells become harder and drier as they move to the surface until the final layers are sloughed off. Required flexibility is maintained by small amounts of water and oils infiltrating up between cells and layers. It becomes obvious that the further bioactive materials can be induced into and down through the layers of the stratum corneum the greater will be the exposure to both the reticulatory and deeper circulatory fluids and subsequent dissolving and pick up of materials by those fluids. Increasing both the amount induced and the surface area of the material exposed to body fluids will also increase its uptake and transport as both these factors are involved.

Further, recent research into skin structure and function has provided insights which demonstrate that present beliefs and methods of transdermal delivery have been based on incorrect assumptions. The incorrect assumptions have resulted in the design of transdermal drug and nutrient delivery systems which are self limiting by the selection of preparations and methods based on these incorrect assumptions.

Understanding the cells and layers of the skin is paramount to an effective transdermal delivery system. In that regard, the cells and layers of the skin include epidermal cells which originate in the basal layer and push up toward more superficial layers. As the epidermal cells move to the surface the cytoplasm, nucleus, and other organelles are replaced by keratin and the cells die, eventually being sloughed off the skin surface. The lower living layers totaling some 100 microns (0.1 mm) in thickness are relatively loosely connected and are protected by the dead outer layer—the stratum corneum, which is about 10 microns (0.01 mm) thick. It is this outer layer, the barrier to entry to the skin, that has not been and is still not completely understood.

As seen in FIG. 1, the outer layer of the skin 100, the stratum corneum, is approximately ten microns thick and consists of some twenty eight layers of dead plate-like cells which are not held together by cell junctions or glued by an intercellular matrix, as previously believed, but are held together instead by protein rivets. Further, dispersed through the stratum corneum are several lipid bilayers. These bilayers consist of layers of long chain fatty acids and ceramides separated by thin water layers held together by elastic ties passing from layer to layer. This water/elastic layer structure is held between two keratinised cells in the form of a sandwich.

Keratinised stratum corneum cells are strengthened by random keratin fibres which restrict any expansion of surface area. Cells normally expand in the presence of water. When stratum corneum cells absorb water they expand up to 25% vertically but, being held by rivets, are severely limited horizontally (2% -3%). This puts pressure on the border areas of the plates, effectively sealing them—a homeostatic reaction designed to close off the spaces between the cells making the skin impenetrable to the further entry of water, or conversely, loss of water.

Keratin requires a constant supply of moisture to remain flexible. Transport of nutrients and water up through the layers of the stratum corneum occurs via the intercellular spaces as the border layers of the cells are almost impermeable to water and oils. The transport of nutrients, required for maintaining the integrity of the system, is between the plates 104 in a wandering random fashion 106.

It appears from FIG. 1 that the riveted plate structure allows a looseness of formation with spaces existing between the layers of flat skin cells and between the borders of adjoining cells. The rivets are tightest in the lower layers with the tension reducing as the cells make their way to the surface until the outer four or five layers are reached. At this stage the cells are hard and dry and some of the rivets begin to let go as the skin enters the sloughing process. The present invention is premised upon the notion that as the bilayers are constructed as a sandwich dimensionally the same as the keratin layers that, rather than being an impenetrable water barrier, the bilayers are specifically present to supply fats, oils and water to the adjacent layers of fully keratinized cells. This is to maintain flexibility in what would otherwise be a hardened leather like structure.

Therefore, the stratum corneum structure provides a dual barrier mechanism designed specifically to prevent both the loss of body fluids and the entry of extraneous fluids. The dual barrier system consists of (a) the border sealing function brought about by the rivets and the horizontal dimensional stability of the cell plates, and (b) the labyrinth seal effect of the 28 cell layers with staggered openings between the cell plates of the system.

The problem of transdermal delivery is to devise a method that will overcome both barriers. Twenty eight layers of cells with a total thickness of 10 microns make up the stratum corneum; that is, each layer is approximately 0.36 ug. Simple arithmetic would suggest that the available spaces between the cells would not allow passage of particles as large as 2 microns. This observation has been the primary reason that researchers have not previously explored or have failed to recognize the possibilities of this pathway. Laboratory trials and then successful trials of both ibuprofen and vitamin C transdermal delivery creams for patients with joint and/or soft tissue injury and inflammation, however, proves that transit of particles through the stratum corneum labyrinth is achieved in spite of this arithmetical improbability. Again, considering the 0.36 ug dimension the obvious conclusion would be that the smaller the particle the better the delivery. This is correct down to size 0.4 ug. Below this, if particles used are of regular size, there is a fall off in delivery until at sizes<0.1 ug the delivery is approximately 5% of the delivery resulting from mixtures with particles sizes noted in the “preferred option”.

The present invention is premised upon the postulate that the flexibility of stratum corneum cells and that stretching of the protein rivets under mechanical pressure combine to accommodate the particle sizes found to pass through the stratum corneum labyrinth. Evidence of the incredible ability of the skin to increase in dimension is clearly demonstrated by the heavy wrinkling of water soaked skin and the expansion occurring during pregnancy.

It is the ignoring of the apparent mathematical absurdity associated with larger particles being able to transit the stratum corneum and the understanding of the protein rivet mechanisms of homeostasis, the ramifications of skin structure and plasticity and the utilization of these factors to selectively deliver biologically active particles into the system that constitute the basis of this invention.

### SUMMARY OF THE INVENTION

The present invention provides a method of delivery and compositions of biologically active agents and carrier compounds which overcome both barriers in a new and novel transdermal or transepithelial delivery system.

According to a first aspect the present invention provides a transdermal composition for administration of a biologically active agent, said composition comprising said biologically active agent and a pharmaceutically acceptable carrier wherein the biologically active agent is present in a form that includes fine solid particles dispersed though the carrier and a substantial proportion of the fine solid particles are sized less than 2 microns.

According to a second aspect the present invention provides a transdermal composition for administration of a biologically active agent, the composition comprising said biologically active agent and a pharmaceutically acceptable carrier wherein said biologically active agent is present in a form including fine solid particles dispersed throughout the carrier and a substantial proportion of the solid particles are sized less than 1 micron. Preferably, at least 80% of the solid particles are sized less than 0.5 micron, even more preferably 80% of the particles are sized less than 0.4 micron with not more than 5% being amphoteric. The most preferred option has a particle range: -0.4 um=20%, 0.3 um=40%, 0.2 um=20%, <0.1 um-20% with amphoteric<5%.

According to a third aspect the present invention provides a transdermal delivery of a microfined biologically active agent via uncoated particles sized less than 0.5 micron without the benefit of a carrier.

According to a fourth aspect the present invention provides a trans epithelial composition for administration of a biologically active agent to the epithelial linings of the body cavities comprising the bronchial system, mouth, oro-nasal passages, vagina and rectum. Transepithelial absorption of microfined particles is facilitated by the absence of keratinised cells and the shape of the mucosal cells of the target epithelia. The two cell types dominant in this tissue are ‘simple columnar’ and stratified squamous cells. These cells form tissue matrices which have larger surface interstices than those found in the stratum corneum. In the case of ciliated tissues the ciliated surface cells are arranged in small clumps with surface voids in between the clumps. Particles are able to fill these voids to then be absorbed.

Massage of epithelial surfaces is obviously impractical, however, there is a recycling of mucous which flows from epithelial cells with a substantial portion being reabsorbed into the cells and bloodstream. This recycling is the major route for absorption of transepithelial microfined particles lodged in the surface of epithelial tissue. A second route of absorption occurs when microfined particles lodged in the surface spaces are carried into the system by endocytosis. The efficacy of the epithelial delivery route is verified by its use in:

1) nasal epithelia—snuff (large particles of tobacco 80-100 ug) to deliver nicotine and the nasal use of the highly soluble drug cocaine.

2) oral epithelia use of buccal and sublingual pathways to deliver both liquid and saliva soluble medication.

3) rectal use of suppositories and liquid induction to deliver soluble medications.

4) GIT—the use of mycelles (oil globules less than 1.0 ug) to deliver vitamin E and A.

In spite of the obvious advantages, the medical literature fails to show any recognition or understanding of the possiblities of particulate delivery of bioactive substances into the mucosa.

According to a fifth aspect, the present invention provides a transdermal composition for administration of a biologically active agent, said composition comprising said biological active agent and a pharmaceutically acceptable carrier wherein the biologically active agent is present in a form that includes microfined solid particles dispersed through the carrier and a substantial portion of these microfined solid particles are less than two microns in size and wherein the composition is essentially free of water and also free of cosmetic ingredients whose mode of action triggers homeostatic border sealing, a mechanism in place to specifically prevent both loss of and absorption of water through the skin.

According to a sixth aspect, the present invention can provide a method of achieving a predetermined programmed rate of percutaneous transfer of a biologically active agent and a transdermal composition for achieving such transfer wherein the particle sizes of the biologically active agent are adjusted to ensure the programmed rate of transfer is achieved. Thus the transdermal composition may contain biologically active agents in a range of particle sizes. The particle sizes may be adjusted to give a relatively constant rate of percutaneous transfer over an extended period up to forty eight hours.

In a further aspect, the present invention provides a method for delivering a biologically active agent transdermally to a body that comprises applying a composition according to the invention to the skin of the body. In a further aspect, the invention provides a method for delivering a biologically active agent transepithelially that comprises applying an active composition to the mucous membranes of the body according to the fourth aspect of the invention. In a particular aspect, the present invention includes a method for the treatment and/or prevention of a disorder in a human or animal which comprises administering topically an effective amount of a composition described herein to the human or animal.

### BRIEF DESCRIPTION OF THE DRAWINGS

FIG. 1 shows the skin layer structure and the transport path of nutrients.

FIG. 2 shows a graph illustrating the absorption of biologically active agents based upon different particle sizes over time.

FIG. 3 shows a graph illustrating the effect that the exclusion of water in the carrier has on transdermal delivery.

### DETAILED DESCRIPTION OF THE PREFERRED EMBODIMENT

According to the present invention the border sealing mechanism of the cell plate and cell layers can be overcome by the exclusion of water and materials that trigger this mechanism.

The mechanism for delivery is mechanical, the governing factors being the shape and fineness of the particles, the size distribution of those particles and the avoidance of carriers that trigger the border sealing mechanism of the plates.

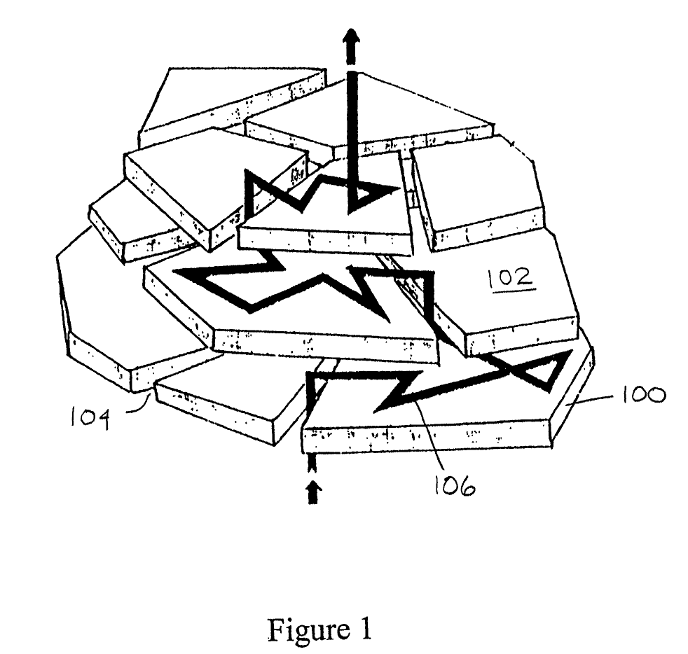

The labyrinth seal effect of the skin was overcome by an unexpected finding that a greatly increased delivery occurred when particles less than one micron were tested. It appears that, while the stratum corneum labyrinth seal works well against liquids, resistance to passage in the intercellular interstices is low enough to allow entrained solid particles to travel between the stratum corneum cells. The wandering flow pattern 106 as seen in FIG. 1, followed by the particles allows the spaces between layers of cells to be filled; the active material is then induced to flow into the next layer by the following entrained material. In this way the very fine material appears to be pressured down through the epidermis. At the same time the filling of the intercellular spaces builds up a reservoir of material subsequently to be absorbed into the system via the reticulatory circuits at a relatively high and constant rate. Another advantage was that particles could also breach the fatty bilayer system which indicates that these layers were not a barrier but served another function—specifically to provide water and oils to keep the keratinised cells flexible.

As the reticulatory circuits flow both out to the surface and back into the system the inventor had assumed there would be a loss of active ingredient back to the surface. Skin tests were carried out using a 40% ascorbic acid composition. After massage the skin was cleaned off with an alcohol wipe and over the next 8 hours the surface was tested for acidity. An unexpected finding was that there was a very small loss of active ingredient back to the surface over the first hour after application after which there was no detectable further loss. Apparently once materials are in the stratum corneum there is a preference for the incoming rather than the outgoing carriage of the reticulatory circuits. This may be due to the polar shunt. A test was carried out by using a hand held massager to induce microfined zinc oxide (as insoluble particles) into a marked section of live udder and the marked section was recovered after slaughter twelve hours later. The recovered tissue was microscopically sectioned and examined. Particles up to size 0.1 ug were found in all levels down to and into the dermal tissues. This finding ran contrary to the inventor's expectation that any actual particle penetration would end at the stratum spinosum. No conclusions have been drawn from this single test. The finding will be the subject of further research.

As previously described, the mechanism for delivery is mechanical, the governing factors being the shape and fineness of the particles, the size distribution of those particles and the avoidance of carriers that trigger the border sealing mechanism of the plates. It was found that particle size reduction below certain limits (essentially defined in preferred option sizes in Aspect 2) decreases the effect of the delivery mechanism and furthermore that if the percentage of amorphic or near amorphic particles climbs above 5% by weight transdermal delivery is reduced. From the test results of various experiments it has been found that there needs to be a certain minimum percentage by weight of particles to bring about entrainment of particles within the skin.

Tests of admixtures with less than 5% particles by weight gave reduced results. To solve this problem, tests for delivery of materials which needed only a small percentages of the active, were carried out using a 5% ibuprofen admixture which included a second particulate material (15% ascorbic acid) as a promoter to provide the mechanical entrainment necessary. Note: any particulate material can promote entrainment.

As particle size decreases the surface area of a material grows rapidly. This has an advantage in that the rate of solubilization of a material is a function of exposed surface area and so the finer particles with a much greater surface area are more easily and rapidly dissolved in body fluids and taken out of the skin reservoir into the system. Another advantage of the present invention is that mixed tocopherols in tested admixtures had a much higher absorption than that found in creams without particles. It appears that carrier liquids trapped in the voids occurring within the particle mass are carried along with the particles increasing delivery of liquid of dissolved actives present in the transdermal admixture.

The present invention considers that the standard Franz Cell test methods provide an inappropriate view of the absorption of tested materials in that the contents of the upper cell remain as a liquid or ointment continuously in contact with the test skin area. The only medical or cosmetic method of transdermal delivery in the market place using this format is patch technology wherein the materials to be absorbed are kept continuously in contact by virtue of an outer impermeable holding film.

Unlike patch technology, creams and lotions are normally applied and then the surplus is wiped off or allowed to air dry. Although current testing procedures do not allow for this common method of use the present invention considers that delivery systems testing of topical applications does not take account of this common practical usage and is therefore essentially irrelevant. To counter this problem, Franz Diffusion Cells using the widely accepted Perfused Bovine Udder Skin model were fitted with vibrators which held a soft plastic head against the test skin in a massaging action. The vibrator was allowed to move randomly over the test sample using its own mass to provide the pressure. The total weight of a vibrator and head was 180 grams and the massage heads were 1.2 cm in diameter. The vibrators provided a massage pressure well below that which would be applied by a person finger massaging cream into the skin.

Testing was done by preparing sample creams as discussed below. Each sample was weighed and spread on the test skin. At this point the sample was subject to five minutes of the massage action described and then the remaining cream was removed and skin surface wiped dry. The weight of the absorbed amount was determined by weighing the removed material. The cell was not capped, the skin being left exposed to the air. Table 1, below, indicates the calculated or determined amount of absorbed cream based upon particle size. Tests were carried out using particles of varying sizes and the following examples chosen as representative of the change in skin loading and delivery rate with reducing particle size. In Table 1, Test a. was carried out with particles sized in the range of 1.0 um plus or minus 0.25 um and Test b. with particles in the range of 0.5 um plus or minus 0.2 urn. Particles for Test c. were obtained by classifying particles in size groups and then combining them in a mixture with the approximate particle sizes in the composition being: −0.4 um=20%, 0.3 um=40%., 0.2 um=20%, <0.1 um=20%. This combination of particle size gave the best 24 hour time/rate delivery of all the tests and is defined as the preferred option for 24 hour duration delivery. While, as noted, it was found that the larger sized particles used in Test a. gave a lower skin loading and rate of delivery, it was also found that the overall constant rate delivery time of that reduced amount was substantially increased. This indicates that varying rates of delivery can be achieved by manipulation of particle sizes. No further specific testing was done in this area. Apparent from Table 1 is that the weight of absorbed cream was improved by reducing the particle size.

TABLE 1

| Test | Particle size | Weight/cm² |
|---|---|---|
| a. | 1 micron ± 0.25 um | 140 micrograms |
| b. | 0.5 micron ± 0.2 um | 355 micrograms |
| c. | 0.4 um 20%, 0.3 um 40%, | 460 micrograms |
| | 0.2 um 20%, <0.1 um 20% | |

Ascorbic acid was chosen as the biologically active agent for the rate/time testing program, as all preliminary research testing could be done by a simple color stick test of the Franz cell buffer solution. Early tests using ibuprofen and magnesium citrate demonstrated absorption characteristics noted were not peculiar to ascorbic acid confirming ascorbic acid as a suitable model. Post program trials on arginine and creatine also showed comparable results. All final tests were carried out with HPLC analysis. Trials with patients experiencing joint pain have demonstrated that transdermally delivered actives are carried on into deeper tissues.

Preliminary test results did not duplicate absorption of larger size particles as found in laboratory rat trials described in U.S. Pat. No. 5,308,621 (Taylor et al.). Using the bovine udder skin model absorption of the ascorbic acid only began to be demonstrated at particle sizes below 7 microns.

One aspect of the present invention provides a transdermal composition for administration of a biologically active agent, said composition comprising said biologically active agent and a pharmaceutically acceptable carrier wherein the biologically active agent is present in a form that includes fine solid particles dispersed though the carrier and a substantial proportion of the fine solid particles are sized less than 2 microns.

For the purposes of this specification the particle size should be determined by microscopic examination and estimation of the largest visible dimension of the particles. Sizes below the point where crystalline structure can no longer be identified by spectroscopy are given the general designation of “amorphic particles.”

A composition may include a single biologically active agent or two or more biologically active agents. It is therefore to be understood that the term “Biologically active agent” also encompasses two or more biologically active agents.

Superior transdermal absorption of actives is achieved if the carrier selected is such that the biologically active agent is only slightly soluble or substantially insoluble in the carrier. However the amount of biologically active agent initially dissolved in the carrier may be in the range of 0.1% to 30%, more preferably 0.1% to 7% by weight; most preferably the biologically active agent will be insoluble in the carrier.

It must be understood that while bioactive particles may be insoluble in the carrier the bioactive agents must be soluble in the cellular fluids for delivery to take place.

By using a combination of small particle size and a carrier in which the biological active is relatively insoluble, the applicant has found that the biologically active agent can be dispersed in the carrier without the need for any foreign coating on the particles. Thus the biologically active agent can be dispersed in the carrier in the form of pure solid particles.

Another aspect of the present invention provides a transdermal composition for administration of a biologically active agent, the composition comprising said biologically active agent and a pharmaceutically acceptable carrier wherein said biologically active agent is present in a form including fine solid particles dispersed throughout the carrier and a substantial proportion of the solid particles are sized less than 1 micron. Preferably, at least 80% of the solid particles are sized less than 0.5 micron, even more preferably 80% of the particles are sized less than 0.4 micron with not more than 5% being amorphic.

The most preferred option of particle sizes for delivery of the actives over the target period of 24 hours has the following approximate size range 0.4 microns 20%, 0.3 microns 40%, 0.2 microns 20%<0.1 micron 20% amorphic not more than 5%.

In addition, it was found that for successful entrainment of particles to occur there needs to be an appropriate spread of particle sizes and there is a limit to useful size reduction of particles. As particle sizes are reduced below the preferred option there is a fall off in delivery. It is particularly noted that amphoteric particles should be kept to below 5% by weight of the materials to be entrained.

Advantageously the amount/rate at which the “reticulatory circuit” mechanisms carry the stored particles into the system also increases with reducing particle size because of the large increase in the surface area of the bioactive material exposed to the body fluids.

It was found that with progressive reduction of average particle size below 1 micron both stratum corneum loading and system delivery were correspondingly further enhanced.

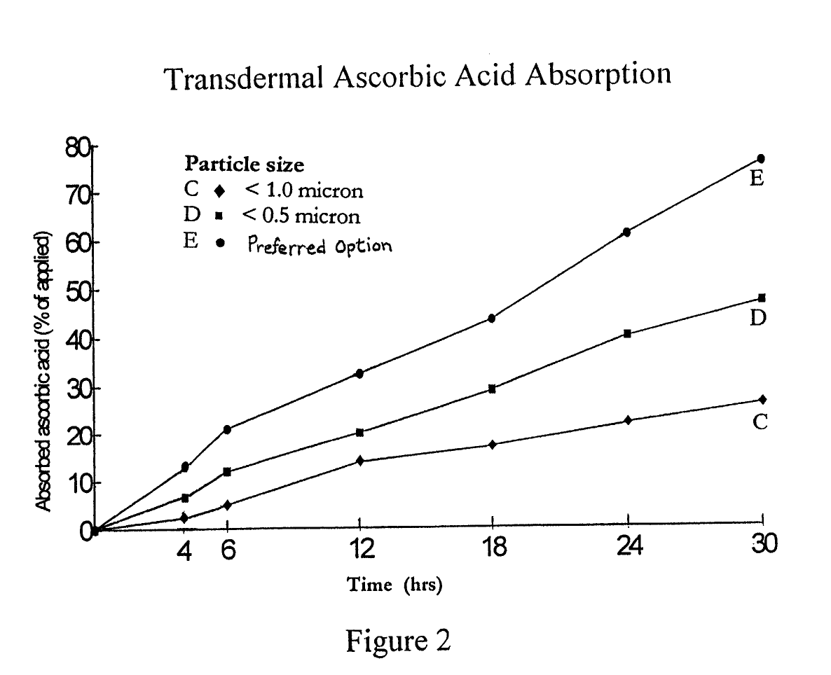

As shown in FIG. 2, reducing the particle size allows the finer particles to penetrate to a much deeper level. This exposes the particles to a larger spectrum and quantity of reticulating and circulating fluids, increases the surface area exposed to the fluids and increases the total weight of material stored in the tissue for ongoing delivery. The ultimate result was delivery of the biologically active material at a high dosage and with a relatively constant time rate delivery over 24 hours (the preferred target delivery period). It has also been found useful to select carrier liquids with characteristics of low viscosity and high slip.

A further aspect of the present invention provides a transdermal delivery of a microfined biologically active agent via uncoated particles sized less than 0.5 micron without the benefit of a carrier.

An unexpected finding was that a powder of similar particle specification to the optimum defined in preferred option particles in Aspect 2 could be massaged directly through the stratum corneum without any material other than the active being involved. In the case of the dry powder delivery the spread of particle sizes was more critical in the success of the entrainment of particles within the stratum corneum. The weight of material that could be induced into the stratum corneum was considerably less than when a carrier was used.

It was also found that milled particles were more readily absorbed than those formed by crystallization. It is assumed that this is due to the more regular shape of the milled materials.

Tests were carried out to determine the maximum of both admixture and dry powder that could be induced into the stratum corneum. The vibrator weight was increased to 400 grams and vibration time to 10 minutes. Results are indicated in Table 2 below. Zinc gluconate (being of particular interest) was used as the active for these tests. There will probably be variations in the optimum particle size range for other actives.

TABLE 2
Maximum achieved massage induced skin load of zinc
gluconate

| | Particle size | Carrier | Maxim um loading |
|---|---|---|---|
| a. | 1 micron ± 0.25 um | Eutanol G | 1.2 micrograms/cm² |
| b. | 0.4 um 20%, 0.3 um 40%, | Eutanol G | 2.3 micrograms/cm² |
| | 0.2 um 20%, <0.1 um 20% | | |
| c. | 1 micron ± 0.25 um | Nil | 0.6 micrograms/cm² |
| d. | 0.4 um 20%, 0.3 um 40%, | Nil | 1.4 mg/cm² |
| | 0.2 um 20%, <0.1 um 20% | | |

A preferred use of the dry powder delivery system is in the healing of wounds, (including surgical), senile, varicose and diabetic ulcers, below breast infections, rashes such as nappy rash, burns, skin infections and eruptions. More specifically using microfined powders of organic and inorganic salts or compounds of zinc, silver, magnesium, copper, selenium etc. used alone or in admixtures with other healing bioactive materials. Where the skin is open (that is there is effectively no stratum corneum) particle sizes<0.4 microns are preferred.

A still further aspect of the present invention provides a transepithelial composition for administration of a biologically active agent to the epithelial linings of the body cavities comprising the bronchial system, mouth, oro-nasal passages, vagina and rectum. While having no keratinised structure mucous epithelia generally have stratified squamous cells with flattened upper cells and deep cuboidal or columnar shapes with accessible intercellular spaces which can readily be transited by particles in the size range less than one micron down to amorphic.

Micellisation, (creating fatty particles of less than one micron in a stable carrier liquid—a method used for the last twenty five years for direct delivery from the GIT), of fats and oils (e.g. micellised vitamin E in a polymoxilated caster oil carrier) has demonstrated the direct gastro intestinal epithelia to bloodstream absorption pathway of fatty globules when brought to sizes in the sub—one micron range. Similarly chylomicrons which range in size up to one micron readily penetrate the lymph vessels. A similar delivery of solid particles is achieved both with and without the assistance of a carrier when solid particles to be delivered are sized less than one micron. It has been found (in tests using hamster intestinal tissue) that reducing size increases the absorption of the active with the best delivery occurring with particles of 0.3 micron to 0.1 micron size. There is no requirement for a spread of particle sizes in transepithelial delivery systems.

As the epithelium under discussion (all epithelia other than skin) has no stratum corneum there is no problem with border sealing and so water is a useful and a logical carrier. Agents may be used that assist suspension of the particles in the liquid carriers involved in epithelial delivery. In liquid epithelial delivery there is no lower limit to the concentration of particulate active in the carrier.

Bronchial system delivery is via a dry powder puffer dispenser or liquid carrier spray, vaginal delivery is by powder impregnated tampons, sponges, disintegrating pessary, gel or cream.

Another aspect of the present invention provides a transdermal composition for administration of a biologically active agent, said composition comprising said biological active agent and a pharmaceutically acceptable carrier wherein the biologically active agent is present in a form that includes microfined solid particles dispersed through the carrier and a substantial portion of these microfined solid particles are less than two microns in size and wherein the composition is essentially free of water. and also free of cosmetic ingredients whose mode of action triggers homeostatic border sealing, a mechanism in place to specifically prevent both loss of and absorption of water through the skin.

Tests based on the knowledge of stratum corneum homeostatic function has found that the inclusion of water in the delivery system greatly reduces the amount and rate of the active material absorbed. Apparently the result of the ready uptake of water by the stratum corneum cells triggers the homeostatic border sealing mechanism. When this occurs the resistance of the system to entrainment of particles is greatly increased.

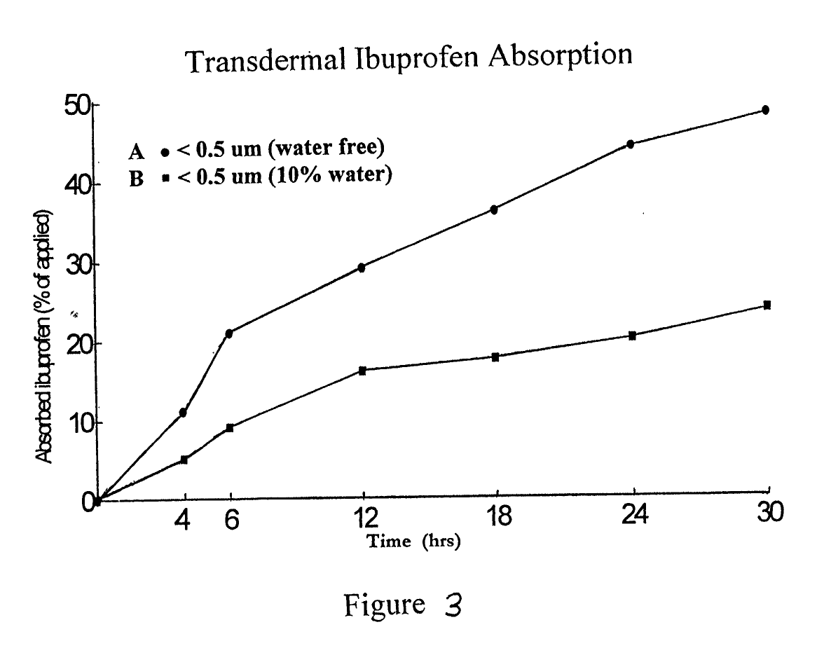

Comparative test results conducted on formulations, as seen in FIG. 3, reveal that the exclusion of water from the formulation resulted in a 100% (approx.) increase in the level of material delivered transdermally.

In addition, acidity is a function of the ionic dissociation of a material in solution. In most circumstances water is the primary dissociation medium and so solutions free of water generally have lower acidity. In an admixture where water is excluded there is low or nil dissociation and it is found that there is no acidity until some water is absorbed into the mixture. While it is impossible to totally exclude water, as there are minute traces in the ingredients utilized as carriers, acidity is kept to a minimum in what are essentially water free admixtures. Water free mixtures used for transdermal delivery have an additional advantage in that biologically active acids such as ascorbic, salicylic, malic, succinic, fumaric citric etc. can be used at higher concentration without creating acid reactions on the skin. For example, it has been found that water based ascorbic acid mixtures can only be used with an ascorbic acid content of 10% to 14% by weight. Ascorbic acid content above 14% by weight has been known to cause burning, stinging and other skin reactions.

Trials of admixtures utilizing water free carriers with ascorbic acid levels as high as 35% were found to be well tolerated by the average consumer with subjective reports of less discomfort than 12% water based admixture.

In experiments conducted with a composition containing ascorbic acid as the active agent, there appeared to be no limit to the concentration of active used in the carrier, as ascorbic acid was trialed up to 45% at which the admixture was almost solid.

For various aspects of the present invention at least 80% of the solid particles of active agent are sized less than 2 microns, more preferably at least 80% of the solid particles are sized less than 1.0 micron, even more preferably 80% of the particles are sized less than 0.5 micron with not more than 5% being amorphic. Most preferably the spread of particle sizes for delivery of the actives over the target period of 24 hours has the following approximate size range: 0.4 microns 20%; 0.3 microns 40%; 0.2 microns 20%; <0.1 microns 20% with amorphic<5%.

The effective rate of transdermal transfer will be dependent on the desired delivery rate and dosage required for the particular biologically active agent concerned. However, in many instances, the compositions would be formulated in such a way as to ensure that at least 40% and up to 70% of the biologically active agent present in the composition would be delivered transdermally within 24 hours of direct application of the composition to the skin depending on the pharmacokinetic characteristics required. When particle size is increased above the preferred option, delivery rate is reduced and the time span of delivery is increased. In this way delivery rate/time can be manipulated.

In another aspect, the invention can provide a method of achieving a predetermined programmed rate of percutaneous transfer of a biologically active agent and a transdermal composition for achieving such transfer wherein the particle sizes of the biologically active agent are adjusted to ensure the programmed rate of transfer is achieved. Thus the transdermal composition may contain biologically active agents in a range of particle sizes. The particle sizes may be adjusted to give a relatively constant rate of percutaneous transfer over an extended period up to forty eight hours.

The compositions may include stabilizers to maintain stability at normal storage temperatures, particularly room temperature, or they may be formulated in such a way as to ensure stability at such temperature.

The delivery system of the invention is particularly useful for delivery of drugs and medications where protection of drug structural integrity, reduction of patient side effects or improved time release characteristics would be served such as anti-anxiety agents, anti-psychotic agents, antidepressants, tranquilizers, beta blockers, angina medications, antihypertensives, antiarhythmic agents, muscle relaxants, diabetic medications, anti-inflammatories, anti-asthmatics, anti-bacterials, anti-virals, anti-fungals, anti-microbials.

The biologically active agent used in the composition is advantageously a pharmaceutically active agent including therapeutic agents, prophylactic agents salts and compounds of metals, herbal extracts, plant extracts, amino acids, peptides, proteins, globulins, hormones, androgenic and estrogenic steroids, sugars, glyco-proteins, vitamins, minerals and drugs.

Other biologically active agents that may be incorporated into the compositions according to the present invention may include but are not limited to the following:

1. Narcotic analgesics—buprinorphine, morphine, methadone, xylocaine.

2. Non-steroidal anti-inflammatories and analgesics—aspirin, acetaminophen, ibuprofen, sodium pentasan polysulphate.

3. Nutritional Compounds and derivatives—enzymes, coenzymes, indoles, anthocyanidins, bioflavinoids, phytohornones, etc.

4. Methyl sulphonyl methane, inositol hexaphosphate, indole-3-carbinol, lactoferrin, cimetidine.

5. Vitamins-C, E, A, B1-2-3-5-6, B12, D, K, folate & their derivatives.

6. Amino acids—arginine, ornithine, proline, creatine, acetyl 1 carnitine, GABA, adenine, aminoguanidine, chondroitin and their derivatives.

7. Sugars, carbohydrates & derivatives—ribose, ribulose, glucosamine galactosamine, mannoheptulose, mannose.

8. Proteins, glycoproteins, lipoproteins, nucleoproteins, peptides, globulins.

9. Herbs, herbal extracts, plant extracts.

10. Minerals & inorganic & organic salts and compounds of metals—zinc, magnesium, potassium, silver, manganese, germanium, lithium, copper & selenium as sulphates, citrates, aspartates, glycerolates, gluconates etc.

11. Antibacterial, antifungal, antiparasitic, antiviral agents embracing plant, animal, and synthetically derived materials.

12. Organic acids—succinic, malic, ascorbic, oxaloacetic acids & their salts and derivatives.

13. Free radical scavengers & anti-oxidants-Co Enzyme Q10, alpha lipoic acid, super oxide dismutase.

14. Anti-psychotics, anti-depressants, tranquilizers, muscle relaxants.

15. Hormones, androgenic and estrogenic steroids and contraceptives—testosterone, dehydroepiandrosterone, estrone, estriol, 4adiol, human growth hormone and their derivatives.

16. Anti asthmatics, anti-hypertensives, anti-arrhythmics, anti hypertensives, angina control.

17. Stimulants—caffeine, amphetamine, nicotine and their derivatives.

18. Dermatological drugs & Antibiotics.

19. Enzyme blocking agents—anastrozole, ricin, nettle extract, saw palmetto extract.

20. Diabetic medications particularly insulin and derivatives.

Any pharmaceutically acceptable derivatives of the foregoing may also be used.

It will be appreciated that the above list is not exhaustive and that the invention also encompasses the use of pharmaceutically active agents other than those specially mentioned.

The term “pharmaceutically acceptable carrier” may include one, two or more carrier agents including both biologically inactive and/or active agents. Examples of pharmaceutically acceptable carriers (otherwise known as vehicles or bases) include glycerols, plant oils, fish and animal oils, proprietary and non-proprietary carriers known to practitioners of the art in the cosmetic and pharmaceutical industry and, where appropriate water. The addition of water is useful in epithelial delivery and where the mechanism of delivery needs to be slowed down and the number of particles entering restricted for medical reasons.

The preferred carriers are inert liquids with minimum viscosity and high slip that do not undergo any significant reaction with or in any way chemically modify the particulate active agent. Biologically active carriers are chosen which do not react with or are not reacted upon by the particulate actives.

It should be appreciated that the above list is not exclusive as the present invention also encompasses the use of pharmaceutically and cosmetically acceptable carriers other than those specifically mentioned. Selection of a carrier is partly determined by the physical and chemical properties of the biologically active agent to be incorporated into the formulation.

Compositions according to the present invention have been found to be suitable for supplying or delivering a biologically active agent for therapeutic and/or prophylactic purposes in both human and veterinary applications.

In still further embodiment the invention involves use of compositions, using the herein before described transdermal preparation arts, in the treatment of a human or animal disorder.

Accordingly, in a further aspect, the invention provides a method for delivering a biologically active agent transdermally to a body that comprises applying a composition according to the invention to the skin of the body. In a further aspect, the invention provides a method for delivering a biologically active agent transepithelially that comprises applying an active composition to the mucous membranes of the body according to the fourth aspect of the invention. In a particular aspect, the present invention includes a method for the treatment and/or prevention of a disorder in a human or animal which comprises administering topically an effective amount of a composition described herein to the human or animal.

In a particularly preferred embodiment the invention involves use of a composition, as hereinbefore described, in the treatment of a localized injury, infection and/or inflammation wherein said active agent is an anti-inflammatory, wound healing or antimicrobial agent and/or any of the other agents or classes of agents disclosed herein.

Compositions of the present invention have been found to be particularly efficacious in delivering the biologically active agent by transdermal migration following topical application and transepithelial migration and these are the preferred routes for administering the compositions.

In this specification, all percentages are given as percentages by weight. Those skilled in the art will appreciate that the present invention is susceptible to variations and modifications other than those specifically described. It is to be understood that the present invention encompasses all such variations and modifications that fall within its spirit and scope.

The following test methods were chosen as a suitably accurate model to assess the potential effects on human and animal skin and epithelia. Perfused bovine udder skin was used as the appropriate model.

### EXAMPLE

In order to demonstrate the present invention, two compositions containing ibuprofen as the active agent were prepared. Particles in both samples were identical; with the particle size less than 0.5 um, however sample, A was water free; while sample B contained 10% water. The samples contained no agents that could cause occlusion of the skin or of the particles. Iboprofen was chosen for this testing as it is relatively insoluble in water. Transdermal absorption of the ibuprofen preparations (sample A and sample B), were compared using fresh bovine udder skin mounted on Franz diffusion cells (FDC) maintained at 37 degrees C.

The recipient chamber of the FDC was filled with a phosphate buffer pH 5.8 containing ethylenediaminetetra-acetic acid (EDTA) and dithiothreitol (CTT) to minimize oxidation of the ibuprofen. The skin was allowed to equilibrate on the FDC for 1 hour.

Approximately 30 mg of the ibuprofen preparation was applied to the skin (area 300 mm²) at time 0 using a small spatula. A vibratory massager as previously described was fitted to the cells after each test mix was placed in the cells. The vibrator was operated for five minutes then removed and the surplus mixture was removed and weighed. In addition, 0.5 ml samples were withdrawn for analysis at 4, 6, 12, 24 and 30 hours. The buffer solution was changed at 6 and 18 hours to minimize the formation of a high concentration gradient in the recipient chamber that may limit absorption. The analyses were done by performance liquid chromatography with a particular modification. Allowance was made for the removal of the 0.5 ml sample in estimating cumulative absorption. Four FDC tests were used to assess each preparation.

The results as seen in FIG. 3, were expressed as the total ibuprofen absorbed over 30 hours calculated as a percent age of the initial amount of ibuprofen applied. The water free sample (A) demonstrated a higher rate of absorption in less time than a similar formulation containing 10% water sample B. In 10% water formulation or sample B the delivery was more than halved and the time rate of the delivery was found to be greatly reduced with delivery curve showing 16% over 12 hrs and only a further 7.5% delivery over the next 12 hours.

Therefore, the present invention at least provides a transdermal composition and a method for making a transdermal composition substantially free of water comprising a biologically active agent in the form of microfined particles, sized less than 2 microns down to less than 0.1 microns, which by massage pressure are mechanically entrained within the interstices of the stratum corneum. Particles less than 0.5 microns do not require a carrier for entrainment. Delivery into mucosal epithelia is obtained by particles less than one micron with delivery increasing with decreasing particle size the preferred size being less than 0.4 microns.

Another aspect of the present invention includes the use of powders, sprays, powder puffers, powder impregnated tampons and dressings, pessaries, disintegrating and foaming tablets and creams and gels for transdermal or transepithelial delivery of the biologically active agent.

Specific examples for the transdermal treatment of osteo arthritis, soft tissue and joint injury, psoriasis, herpes, skin cancer, male hormone replacement, penile erection, anti-aging, mitochondrial energy, topical anesthetics, ulcers and radiation damage using the present invention are discussed in detail below.

Biophysical and biochemical damage and its repair is always multifaceted be it in injury, degeneration or infection. The need is to develop protocols where each of the metabolic pathways involved in repair, healing and patient comfort are joined in a treatment whereby the necessary bioactive agents are delivered together at the right place and in the right ratio. Within the constraints posed by the digestive system this is rarely possible via the gastrointestinal, route.

Osteo Arthritis

Osteo arthritis and psoriasis have been dealt with in some detail as examples of the complexity of the treatment pathways. Other transdermal protocols and treatments are given in lesser detail. For osteo arthirits there is a need to: (1) stop the breakdown of collagen and proteoglycan by tumour necrosis factor alpha (TNF-a) and interleukin one beta(IL-lb); (2) promote proteoglycan, hyaluronic acid and collagen production and increase the water and ground substance in the proteoglycan matrix; (3) decrease inflammation; (4) increase cellular energy production; (5) induce a load bearing lubricant into the joint; (6) promote blood flow to the surrounding tissues; and (7) reduce pain.

The Transdermal approach using the present invention disclosed would be to:

a. Inhibit TNF-a and IL-lb; Nettle leaf extract potently inhibits the genetic transcription factor NF-kb that activates TNF-a and IL-lb in synovial tissue.

b. Glucosamine sulphate is used by the body to synthesize the proteoglycans and the water binding glycosaminoglycans (GAGs) in the cartilage matrix. Chondroitin sulphate is a major component of cartilage, which attracts water into the cartilage matrix and stimulates the production of cartilage, galactosamine stimulates hyaluronic acid production. Vitamin B5 (pantothenic acid) is a proteoglycan promoter. Methyl sulphonyl methone (MSM) helps the production of ground substance, which keeps connective tissue intact and enhances the structure of mucopolysaccharides found in high concentration in cartilage. Collagen type 2 contains the most important compounds for connective tissue repair and production of new cartilage. Ascorbic acid is essential to the absorption of collagen type 2 and for the manufacture of collagen within the joint.

c. Ginger extracts and Aloe Vera inhibit cyclooxygenase and lipoxygenase and the production of prostaglandins, thromboxane and leukotrienes thereby reducing inflammation.

Gamma aminobutyric acid (GABA) inhibits cyclooxygenase (COX-2), Omega 3 fatty acids suppress leukotriene B(4) and aggreconases in the joint and increases production of anti inflammatory prostaglandin-1 mediated products. Alpha lipoic acid, vitamins E, A and D all have anti-inflammatory properties.

d. Coenzyme Q10, alpha lipoic acid, ribose and acetyl 1 carnitine all act synergistically to increase cellular energy production.

e. Sodium pentasan polyphosphate and cetyl myristate act as joint lubricants.

f. Niacinamide or tocopherol nicotinate increase blood supply to deep tissues.

e. Cobalamin, folate and niacin reduce joint pain and increase mobility. The following formulation components constitute the preferred actives for inclusion in an osteo arthritis treatment cream.

The preferred formulation would include: nettle leaf extract, glucosamine sulphate, chondroitin sulphate, galactosamine, pantothenic acid, MSM, collagen type 2, ascorbic acid, ginger extract, aloe vera, GABA, alpha lipoic acid, vitamins E, A and D, CoQ10, ribose, acetyl 1 carnitine, cetyl myristate, niacinamide, cobalamin, folate and niacin, to be incorporated into a carrier consisting of omega 3 fatty acid, almond oil, carrot oil and cosmetic oils, waxes, anti oxidants and anti microbials as used regularly in the cosmetic industry. Sodium pentosan polyphosphate may be included in the formulation when its use is approved by the FDA.

Soft Tissue and Joint Injury

The healing needs for this product are similar to those for osteo arthritis. The difference is in the need to suppress biological triggers that originate arthritis and in the need to repair torn tissue in soft tissue and joint injury.

The preferred formulation would include: taurine, cysteine, urea, glucosamine sulphate, pantothenic acid, MSM, collagen type 2, ascorbic acid, aloe vera, alpha lipoic acid, vitamins E, A, D CoQ10, ribose, cetyl myristate, niacin, BHT, conjugated linoleic acid, grape seed extract to be incorporated into a carrier consisting of omega 3 fatty acid, almond oil, carrot oil and cosmetic oils, waxes, anti oxidants, anti microbials as used regularly in the cosmetic industry. Sodium pentosan polyphosphate may be included in the formulation when its use is approved by the FDA.

Psoriasis

Psoriasis patients exhibit increased cyclic guanosine monophosphate (cyc GMP) induced cell proliferation. Decreased cyclic adenisine monophosphate (cyc AMP) induced decreased cell maturation and increased cell proliferation (cyclic GMP and AMP are energy substrates required to maintain cellular energy levels). High copper, low zinc levels, excessive arginine and ornithine related polyamines, low essential fatty acids (EPA's), high arachadonic acid levels with inflammation at site, angiogenisis at lesion site.

The logical route to directly reversing the metabolism responsible for the psoriasis symptoms is to get the required balancing nutrients into cells at the site.

Therefore the Nutraceutical protocol for Psoriasis would be incorporated in a transdermal cream to: decrease cyclic GMP with cysteine, vitamin A, vitamin E, and selenium; increase cyclic AMP with caffeine; inhibit polyamines with vitamin A; block arachadonic acid cascade with EPA (eicosapentaenoic acid); selectively chelate excess copper with malic acid; regulate proliferation and differentiation of cells with Vitamin D3; stimulates production of ground substance and normalise cell metabolism with methyl sulphonyl methone (MSM); and increase cell energy production with Alpha lipoic acid, and ribose.

The preferred formulation the generalized formulation for a psoriasis cream would include: Cysteine, vitamin A, vitamin E, selenium, caffeine, EPA, malic acid, vitamin D3, Alpha lipoic acid, and ribose in a carrier consisting of macadamia oil and published cosmetic ingredients.

Herpes

Herpes infections including oral, genital and shingles (HSV-1, HSV-2, HHV-3) would require treatment to stop viral replication using Cemitidine, Butylated hydroxy toluene (BHT), 1-lysine, citrus bioflavinoids, and quercetin. To repair nerve sheaths the treatment may include methyl cobalamin and acetyl 1 carnitine and to heal internal and external lesions the treatment may include threonine.

The preferred formulation would include: Cemitidine, L-lysine, citrus bioflavinoids, quercetin, methyl cobalamin, acetyl 1 carnitine, threonine, BHT, vitamins A, B, and B5 in a carrier of almond oil and published cosmetic ingredients. This formulation may be made without the inclusion of cemitidine until this drug is approved by the FDA for this purpose.

Skin Cancer

The need in the treatment of pre-cancer and early stage skin cancer conditions is to: (1) inhibit the cells uptake of glucose; (2) provide increased cellular energy to reverse the cell transformation to the glycolysis cycle; (3) retard angiogenesis; (4) inhibit cell reproduction; (5) inhibit beta-glucuronidase enzyme; and (6) strengthen the resistance of the normal cells in the immediate area.

The preferred formulation would include: Taurine, histidine, threonine, creatine monohydrate, quercetin, citrus bioflavinoids, D-glucarate, vitamins C, D3, E, B5, A, folic acid, indole 3 carbinol, CoQi10, alpha lipoic acid, acetyl 1 carnitine, lactoferrin, avocado extract, collagen 2, conjugated linoleic acid, gamma linoleic acid, butyric acid, urea and squaline in a carrier of macadamia/carrot oil and cosmetic ingredients.

Male Hormone Replacement and Anti-Aging

Hormone creams used in therapy are common as the hormone molecule is readily absorbed. The absorption of some crystalline steroids, associated amino acids and enzyme blocking agents cannot be achieved in the same way as hormones yet the need to be delivered and available in the system at the same time is important. The delivery should also include supporting steroids, amino acids, and enzyme blocking agents.

Further, the use of testosterone as an anti aging therapy is adversely effected by: the conversion of free testosterone to estradiol via the enzyme aramatase; the decline in the liver P450 clearance system; and by the need to deliver the hormone in a manner mimicking the body's diurnal release pattern and the need to provide balancing natural anabolic sterols and steroids.

The preferred male hormone formula includes: Testosterone, dehydroepiandrosterone (DHEA), estriol, threonine, guanisine, anastrozole, folic acid, and vitamin C in a carrier of cholesterol, macadamia/grapeseed oil and cosmetic ingredients.

The preferred complete anti aging formulation includes the addition of human growth hormone and acetyl 1 carnitine to the male hormone formula.

A female formulation is based on the same principles as the male and also with the addition of HGH and acetyl 1 carnitine.

Penile Erection

A male erection depends on the liberation of nitric oxide (NO) in the penile tissues to relax arterial smooth muscle structure of arteries allowing blood engorgement of erectile chambers. The life of NO released within tissues is some 5-10 seconds. This makes it essential to: (1) have the release where it is needed and (2) maintain the supply and attenuate the resultant signals. There is also a need to block noradrenaline stimulation of a-adrenergic receptors if arterial relaxation is to proceed.

While a number of chemicals have molecules which will provide the necessary NO the problem remains getting the NO to release into the target tissues. Several companies have proposed the use of arginine (a well known source of NO), however, it appears that the desired chemical release of NO from arginine has not been solved. The present preferred formulation produces the required NO at site.

The preferred formulation includes: Stabilised glycerol trinitrate, ornithine, cyclic guanosine monophosphate, folic acid, amino guanidine, vitamin C, and yohimbine in a carrier of macadamia/apricot oils and cosmetic ingredients.

Detumescent is the rapid reduction of an erection which requires the reversal of erectile function. The preferred formula for initiating detumescence includes: cyclic adenisine monophosphate, glutamine, and aspirin in a carrier of grapeseed oil and cosmetic ingredients.

Anti Aging Cosmetic Cream

In an anti aging creme the need is to: (1) increase mitochondrial energy output using CoQ10, Alpha Lipoic acid, ribose, vitamins B2, B3, C, D3, K2, and folate; (2) provide amino acids for correct cellular production using arginine, proline, taurine, threonine, acetyl 1 carnitine, and guanosine; (3) inhibit formation of lipofuscin and ceroid using lactoferrin and avocado extract; (4) provide fatty acids for cell function using Squaline and GLA; and (5) provide a strong free radical using scavenger grape seed extract.

The preferred formulation includes: arginine, proline, taurine, threonine, guano sine, collagen 2, CoQ10, Alpha Lipoic acid, acetyl 1 carnitine, ribose, vitamins B2, B3, C, D3, K, folic acid, lactoferrin, and avocado extract, in a carrier of squaline, GLA, grape seed extract and appropriate cosmetic ingredients.

Mitochondrial energy

A fall off in mitochondrial energy output is the initiator of many cellular changes which are essentially the start of disease. It is also an advantage in many situations to increase cellular energy output to increase performance (eg. chronic fatigue syndrome).

The combination of Co Enzyme Q10, alpha lipoic acid, acetyl 1 carnitine, ribose and creatine phosphate used as a mitochondrial energy producer is both a stand alone formulation and a combination block for addition for many proposed products.

The preferred formulation includes: Co Enzyme Q10, alpha lipoic acid, acetyl 1 carnitine and d-ribose, and creatine monohydrate in an oil based carrier with cosmetic ingredients.

Topical Anesthetics

Topical delivery of anesthetics using oil soluble drugs has been available for many years.

The need has been to: (1) deliver non-oil soluble drugs; (2) extend the duration of the anesthetic effect; and (3) to carry chemicals that hold the drug longer in the target tissue.

The preferred formulation includes: buprinorpkine, 1-lysine in carrier of apricot oil, cholesterol and cosmetic ingredients.

Ulcers

Ailments such as ulcers, geriatric, varicose, diabetic, bed sores etc. the need is to prevent microbial infection, increase cellular propagation, reduce inflammation, inhibit hyaluronidase, stimulate local circulation, and increase cell energy output.

The preferred formulation (as powder) would include: arginine, threonine, glucosamine, urea, zinc glycerolate, magnesium laureth sulphate, aloe vera, vitamin A, C, D3, B5, E succinate, 1C3, Ginseng extract, and echinacea extract in a carrier of oils and common cosmetic ingredients.

Radiation Damage and Repair

All forms of radiation X-rays, gamma rays, or Ultra Violet light damage tissue via the same mechanisms. To repair and prevent damage the formula should reduce lipid peroxidation, protein oxidation, free radical generation, inflammation, DNA/RNA cross linking, degradation of cell membranes, and inhibition of prostacyclin (PGI2) and other hormones.

The preferred formulation in a sunscreen would include: glutamine, arginine, histidine, taurine, MSM, gotu kola extract, ginseng extract, quercetin, BHT, aloe vera, vitamins A, E, C, K1, titanium dioxide in a carrier of macadamia oil, almond oil, CLA, squaline and cosmetic ingredients.

Although the foregoing invention has been described in some detail by way of illustration, examples, and preferred formulations for purposes of clarity of understanding, it will be obvious that certain changes and modifications may be practiced within the scope of the appended claims.

What is claimed is:

1. A transdermal composition for transdermal administration of a biologically active agent comprising:
at least one biologically active agent;
a pharmaceutically acceptable carrier;
wherein said at least one biologically active agent is induced into the skin by massage and includes a plurality of fine solid particles sized less than 2 microns dispersed through said carrier; and
wherein said transdermal composition is substantially free of water.

2. A method for delivering a biologically active agent via transdermal or transepithelial delivery comprising the steps of:
applying an active agent composition substantially free of water to at least one application region; and
wherein said active agent composition is comprised of at least one biologically active agent and a pharmaceutically acceptable carrier;
wherein said at least one biologically active agent includes a plurality of fine solid particles sized less than 2 microns dispersed through said carrier; and
massaging said active agent composition into said application region consisting of a skin region.

3. The method of claim 2, further comprising the step of applying said active agent composition onto said application region for transepithelial delivery wherein said application region consists of the mucous membranes of the gastro intestinal system, the respiratory system, nasal cavities, the vagina, or rectum.

4. The transdermal composition of claim 2, wherein said transdermal composition is in the form of an ointment.

5. The transdermal composition of claim 2, wherein said transdermal composition is in the form of a cream.

6. The transdermal composition of claim 2, wherein said at least one biologically active agent is partially soluble in the pharmaceutically acceptable carrier.

7. The transdermal composition of claim 2, wherein said at least one biologically active agent is substantially insoluble in said pharmaceutically acceptable carrier.

8. The transdermal composition of claim 2, wherein said transdermal composition is substantially free of water.

9. The transdermal composition of claim 8, wherein said transdermal composition minimizes acid skin reactions resulting from normally acid ingredients.

10. The transdermal composition of claim 2, wherein a range of between 30% and 100% of said plurality of fine solid particles are sized less than 1 micron.

11. The transdermal composition of claim 2, wherein a range of between 30% to 100% of said plurality of fine solid particles are sized less than 0.5 micron.

12. The transdermal composition of claim 2, wherein said at least one biologically active agent is induced into the skin by massage.

13. The transdermal composition of claim 2, wherein said plurality of fine solid particles are approximately comprised of fine solid particles with 20% sized at 0.4 microns, 40% sized at 0.3 microns, 20% sized at 0.2-0.1 microns, 10% sized less than 0.1 micron, and 5% being amorphic.

14. The transdermal composition of claim 2, wherein a composition of said plurality of fine solid particles is selected to provide desired pharmacokinetic properties in said transdermal composition.

15. The transdermal composition of claim 2, further comprising a plurality of microfined particles free of carrier wherein said plurality of fine solid particles fall within a range from 0.5 micron to amorphic.

16. The transdermal composition of claim 2, wherein said plurality of fine solid particles are sized at about 0.3 microns.

17. The transdermal composition of claim 2 wherein said at least one biologically active agent is selected from the group consisting of sedatives, analgesics and narcotic analgesics, non-steroidal anti-inflammatory drugs and agents, anti-psychotics, anti-depressants, tranquilizers, muscle relaxants, nutritional compounds, anti-oxidants and free radical scavengers, vitamins, mineral salts, organic and inorganic salts of metals, amino acids, proteins, glycoproteins, lipoproteins, nucleoproteins, peptides, globulins, sugars, herbal extracts, antibacterial, antifungal and antiviral agents, corticosteriods, local anesthetics, antihistamines, androgenc and estrogenic steroids and contraceptives, hormones, anti-asthmatic agents, dermatological disorder drugs, antibiotics, diabetic medication, stimulants, organic acids and chemical or herbal blockers of enzymes such as aromatase.

18. The transdermal composition of claim 2, wherein said pharmaceutically acceptable carrier is selected from the group consisting of glycerols, plant oils, fish and animal oils, proprietary and non-proprietary carriers known to practitioners of the art in the cosmetic and pharmaceutical industry.

19. The transdermal composition of claim 2, wherein said pharmaceutically acceptable carrier contains water where required for pharmacokinetics or delivery of the active material.

20. An active agent composition for the transdermal treatment of a condition via transdermal or transepithelia delivery comprising:
at least one biologically active agent for the treatment of said condition; and
a pharmaceutically acceptable carrier;
wherein said active agent composition is substantially free of water and said at least one biologically active agent includes a plurality of fine solid particles sized less than 2 microns dispersed through said pharmaceutically acceptable carrier.

21. The active agent composition of claim 20, wherein said condition is osteo arthritis, said at least one biologically active agent is comprised of: nettle leaf extract, glucosamine sulphate, galactosamine, pantothenic acid, MSM, collagen type 2, ascorbic acid, ginger extract, aloe vera, GABA, alpha lipoic acid, vitamins E, A and D, CoQ10, ribose, acetyl 1 carnitine, cetyl myristate, niacinamide, cobalamin, folate and niacin; and
wherein said pharmaceutically acceptable carrier is comprised of omega 3 fatty acid, almond oil, carrot oil and cosmetic oils, waxes, anti oxidants, anti microbials, taurine, and cystine.

22. The active agent composition of claim 20, wherein said condition is soft tissue and joint injury, said at least one biologically active agent is comprised of: taurine, cysteine, urea, glucosamine sulphate, pantothenic acid, MSM, collagen type 2, ascorbic acid, aloe vera, alpha lipoic acid, vitamins E, A, D CoQ10, ribose, cetyl myristate, niacin, BHT, conjugated linoleic acid, and grape seed extract; and
wherein said pharmaceutically acceptable carrier is comprised of omega 3 fatty acid, almond oil, carrot oil and cosmetic oils, waxes, anti oxidants, and anti microbials.

23. The active agent composition of claim 20, wherein said condition is psoriasis, said at least one biologically active agent is comprised of: Cysteine, vitamin A, vitamin E, selenium, caffeine, EPA, malic acid, vitamin D3, MSM, Alpha lipoic acid, CoQ10 and ribose; and
wherein said pharmaceutically acceptable carrier is comprised of macadamia oil and common cosmetic ingredients.

24. The active agent composition of claim 20, wherein said condition is herpes, said at least one biologically active agent is comprised of: Cemitidine, L-lysine, citrus bioflavinoids, quercetin, methyl cobalamin, acetyl 1 carnitine, threonine, BHT, vitamins A, B, and B5; and
wherein said pharmaceutically acceptable carrier is comprised of almond oil and common cosmetic ingredients.

25. The active agent composition of claim 20, wherein said condition is skin cancer, said at least one biologically active agent is comprised of: Taurine, histidine, threonine, creatine monohydrate, quercetin, citrus bioflavinoids, D-glucarate, vitamins C, D3, E, B5, A, folic acid, indole 3 carbinol, CoQ10, alpha lipoic acid, acetyl 1 carnitine, lactoferrin, avocado extract, collagen 2, conjugated linoleic acid, gamma linoleic acid, butyric acid, urea, and squaline; and
wherein said pharmaceutically acceptable carrier is comprised of macadamia oil, carrot oil, and common cosmetic ingredients.

26. The active agent composition of claim 20, wherein said condition is male hormone replacement and anti-aging, said at least one biologically active agent is comprised of: Testosterone, dehydroepiandrosterone (DHEA), estriol, threonine, guanisine, anastrozole, folic acid, and vitamin C; and
wherein said pharmaceutically acceptable carrier is comprised of cholesterol, macadamia oil, grape seed oil and common cosmetic ingredients.

27. The active agent composition of claim 20, wherein said condition is penile erection, said at least one biologically active agent is comprised of: Stabilized glycerol trinitrate, ornithine, cyclic guanosine monophosphate, folic acid, amino guanidine, vitamin C, and yohimbine; and
wherein said pharmaceutically acceptable carrier is comprised of macadamia oil, apricot oil and common cosmetic ingredients.

28. The active agent composition of claim 20, wherein said condition is anti-aging, said at least one biologically active agent is comprised of: arginine, proline, taurine, threonine, guanosine, collagen 2, CoQ10, Alpha Lipoic acid, acetyl 1 carnitine, ribose, vitamins B2, B3, C, D3, K, folic acid, lactoferrin, and avocado extract; and
wherein said pharmaceutically acceptable carrier is comprised of squaline, GLA, grape seed extract and common cosmetic ingredients.

29. The active agent composition of claim 20, wherein said condition is Mitochondrial energy, said at least one biologically active agent is comprised of: Co Enzyme Q10, alpha lipoic acid, acetyl 1 carnitine and d-ribose, and creatine monohydrate; and
wherein said pharmaceutically acceptable carrier is comprised of an oil based carrier with common cosmetic ingredients.

30. The active agent composition of claim 20, wherein said condition is topical anesthetic, said at least one biologically active agent is comprised of: buprinorphine, 1-lysine; and
wherein said pharmaceutically acceptable carrier is comprised of apricot oil, cholesterol and common cosmetic ingredients.

31. The active agent composition of claim 20, wherein said condition is ulcers, said at least one biologically active agent is comprised of: arginine, threonine, glucosamine, urea, zinc glycerolate, magnesium laureth sulphate, aloe vera, vitamin A, C, D3, B5, E succinate, 1C3, Ginseng extract, and echinacea extract; and
wherein said pharmaceutically acceptable carrier is comprised of oils and common cosmetic ingredients.

32. The active agent composition of claim 20, wherein said condition is radiation damage, said at least one biologically active agent is comprised of: glutamine, arginine, histidine, taurine, MSM, gotu kola extract, ginseng extract, quercetin, BHT, aloe vera, vitamins A, E, C, K1, titanium dioxide; and
wherein said pharmaceutically acceptable carrier is comprised of macadamia oil, almond oil, CLA, squaline and common cosmetic ingredients.
